(19) United States
(12) Patent Application Publication
(10) Pub. No.: US 2020/0345625 A1
(43) Pub. Date: Nov. 5, 2020

(54) Nanopore Delivery Device

(71) Applicant: Lee Edward Hudson, Elgin, TX (US)

(72) Inventor: Lee Edward Hudson, Elgin, TX (US)

(21) Appl. No.: 16/862,694

(22) Filed: Apr. 30, 2020

Related U.S. Application Data
(60) Provisional application No. 62/842,833, filed on May 3, 2019.

(51) Int. Cl. A61K 9/00; A61K 31/566; A61K 31/568; A61K 31/57; A61K 47/44; B01D 71/52; B01D 71/20; B01D 71/16; B01D 71/48; B01D 71/50; B01D 71/56; B01D 71/02

(52) U.S. Cl. CPC A61K 9/0024; A61K 31/566; A61K 31/568; A61K 31/57; A61K 47/44; B01D 71/52; B01D 2325/02; B01D 71/16; B01D 71/48; B01D 71/50; B01D 71/56; B01D 71/025; B01D 71/022; B01D 71/20

(57) ABSTRACT

The invention relates to an implantable device to deliver drug formulations through a nanoporous membrane. The current related arts for delivery of drug formulations include tablets, injections, implantable pellets, injectable polymer depots, and implantable infusion pumps. The invention employs a reservoir to contain the drug formulation, a nanoporous membrane, and a formulation of estrogen.

### BACKGROUND INFORMATION

The current related arts for delivery of drug formulations include tablets, injections, implantable pellets, polymer depots, and implantable infusion pumps. Recent advances in manufacturing technologies have enabled certain structures to be created at the nanoscale. The present invention of an implantable nanopore device consists of a reservoir, a nanoporous membrane, and a drug formulation of estrogen.

### SUMMARY

Nanoscale porous structures have been used to control the diffision of different types of molecules. The use of nanopores to regulate the diffusion of drugs has been a recent development due to the advancement of nanoscale manufacturing techniques. The present invention uses a nanoporous membrane to deliver a drug formation in a subcutaneous implant.

In U.S. Pat. No. 8,480,637 B2 Nanochanneled Device and Related Methods, a drug delivery system is described wherein the nanoporous part is a silicon wafer based nanochannel device. Semiconductor manufacturing techniques are used to create microchannels and nanochannels from a silicon wafer. The manufacturing techniques are described to create highly precise microchannel and nanochannel sizes.

In U.S. Pat. No. 8,632,510 B2 Nanochanneled Device and Related Methods a drug delivery system is described wherein the nanoporous part is a silicon wafer based nanochannel device. Semiconductor manufacturing techniques are used to create microchannels and nanochannels in a silicon wafer.

IN U.S. Pat. No. 9,005,185 B2 Nanochanneled Device and Related Methods a drug delivery system is described wherein the nanoporous part is a silicon wafer based nanochannel device. Semiconductor manufacturing techniques are used to create microchannels and nanochannels in a silicon wafer.

In U.S. Pat. No. 9,526,824 B2 Nanochanneled Device and Related Methods a drug delivery system is described wherein the nanoporous part is a silicon based nanochannel device. Semiconductor manufacturing techniques are used to create microchannels and nanochannels in a silicon wafer.

In U.S. Pat. No. 10,369,340 B2 Device and Method for Sustained Release of Low Water Solubility Therapeutic Agent in Solubilizer a drug delivery system is described wherein the nanoporous part is a silicon wafer based nanochannel device. Semiconductor manufacturing techniques are used to create microchannels and nanochannels in a silicon wafer and the processes to manufacture are included. The nanochannel part is used in conjunction with a drug formulation which included a solubilizing agent.

In U.S. Pat. No. 5,035,891 Controlled Release Subcutaneous Implant a device is described as a reservoir containing an an amount of a solid hydrophilic polymer sufficient to cause swelling of the device by osmotic pressure after implantation. Drug is forced from the device by the swelling of the internal polymer.

U.S. Pat. No. 8,603,076 B2 Microfabricated Nanopore Device for Sustained Release of Therapeutic Agent a drug delivery system is described wherein the nanoporous part is a silicon wafer based nanochannel device. Semiconductor manufacturing techniques are used to create microchannels and nanochannels in a silicon wafer. Also included is a description of the inclusion of a osmotic material to swell internally to aid in drug delivery.

U.S. Pat. No. 5,152,997 Method and device for transdermally administering testosterone across nonscrotal skin at therapeutically effective levels describes a patch device which delivers testosterone transdermally.

U.S. Pat. No. 5,837,276 Apparatus for the delivery of elongate solid drug compositions describes a hollow cylinder type device containing a solid drug formulation which dissolved from the open end of the cylinder with no type of membrane between the drug formulation and the subcutaneous tissue.

The present preferred embodiment consists of a reservoir capsule, a nanoporous membrane, and an estrogen formulation. The reservoir capsule of the preferred embodiment can be constructed of a biocompatible material such as titanium, titanium alloys, stainless steel 316L, polyetheretherkeytone (PEEK), and polysulfone (PSU).

The nanoporous membrane in the preferred embodiment consists of biocompatible materials manufactured in such a way to create a nanoporous structure of the entire membrane. The specified nanopore size is a distribution of size dimensions around an average pore size with the distribution dependent on the manufacturing technique. Porous titanium is manufactured through a sintering process. One process is raw titanium powder undergoes cold isostatic pressing then sintering at high temperature. The sintered titanium is then vacuum machined to the final shape such as a disc. Stainless steel 316L may also sinter processed to create a nanoporous membrane. The process results in uniform structure, narrow pore size distribution, high separation efficiency and very high porosity.

The preferred embodiment of the nanoporous membrane can be fabricated certain biocompatible materials such as of titanium, titanium alloys, stainless steel 316L, polyetheretherkeytone (PEEK), polysulfone (PSU), aluminum oxide, zirconium oxide, nylon, polycarbonate, cellulose acetate, and cellulose nitrate.

The preferred embodiment of the estrogen formulation may be composed of the following types of estrogen: estrogen powder, estriol, estradiol, estradiol enanthate, ethinyl estradiol, estrone, estrogen sulfate.

The preferred embodiment of the estrogen formulation may also include the addition of the following types of progesterone: progesterone, progestin.

The preferred embodiment estrogen formulation may consist of the following testosterone formulations: testosterone, testosterone enanthate, testosterone decanoate, testosterone cypionate, testosterone isocaproate, testosterone phenylpropionate, and testosterone propionate.

The estrogen formulation in the preferred embodiment may consists of an estrogen formulation mixed with an oil such as: soybean oil, corn oil, olive oil, castor oil, sesame oil, light mineral oil, heavy mineral oil, coconut oil, and canola oil, or other biocompatible oils.

### BRIEF DESCRIPTION OF THE DRAWINGS

FIG. 1 shows a side view cutaway section of the assembled implantable device filled with a drug formulation.

FIG. 2 shows a top down view of the assembled implantable device.

FIG. 3 shows an exploded cutaway view of the parts of the implantable device.

FIG. 4 shows top town view of a nanoporous membrane.

### DETAILED DESCRIPTION OF EXEMPLARY EMBODIMENTS

Referring to the figures, the preferred embodiment of the invention consists of a reservoir housing, a nanoporous membrane, and a formulation of a estrogen.

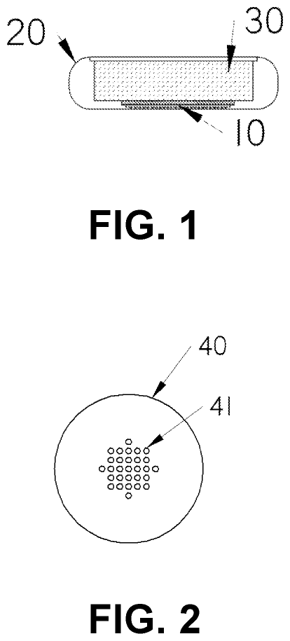

FIG. 1 shows a side view cut away section of a capsule assembly with a nanoporous part 10 assembled to a reservoir body 20 and filled with a drug 30 in combination with an oil. Part 20 comprises the reservoir housing of the invention which can consist of titanium, titanium alloy, polyetheretherketone (PEEK), polysulfone (PSU), 316L stainless steel, or other biocompatible metal or plastic. Part 20 assembled contains a hollow section with volume equal to the amount of drug formulation to be delivered for a desired treatment duration. When the preferred embodiment is assembled and filled with an estrogen formulation and implanted subcutaneously in the human body, the drug formulation passes through the porous membrane into the subcutaneous tissue.

FIG. 2, a top view of part 40 shows holes 41 that the drug formulation exits from the inside of the capsule and from the nanoporous membrane.

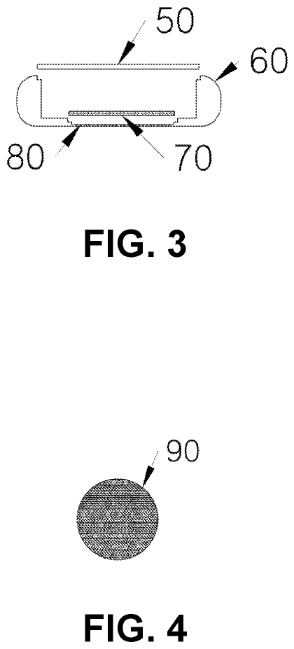

FIG. 3 shows an exploded cutaway of the main capsule body 60, the nanoporous membrane 70, a capsule lid 50 and drilled exit holes 80. The nanoporous membrane 70 attaches to the capsule reservoir part 60 to ensure estrogen formulation placed inside the finished capsule diffuses through the nanoporous membrane 70 and out the holes 80 of capsule part 60 and into the subcutaneous tissue of the body.

FIG. 4 is a top down view of an example nanoporous membrane 90.

The reservoir part 60 may have a fill port opening and a plug or septum installed to allow filling and sealing of an assembled capsule with the drug formulation 30.

The nanoporous membrane 70 is approximately 0.010″ to 0.050″ thick and made of nanoporous material with an average pore sized of 3 nm to 500 nm. The nanoporous membrane 70 can be made of implant grade titanium, titanium alloys, 316L stainless steel, polyetheretherketone (PEEK), polysulfone (PSU), aluminum oxide, zirconium oxide, nylon, polyester, cellulose acetate, cellulose nitrate, and other biocompatible materials that can be manufactured with nanoscale porosity.

The estrogen formulation 30 may be composed of the following types of estrogen: estrogen powder, estriol, estradiol, estradiol enanthate, ethinyl estradiol, estrone, estrogen sulfate.

The estrogen formulation 30 may also include the addition of the following types of progesterone: progesterone, progestin

The estrogen formulation 30 can consist of the following testosterone formulations: testosterone, testosterone enanthate, testosterone decanoate, testosterone cypionate, testosterone isocaproate, testosterone phenylpropionate, and testosterone propionate.

The estrogen formulation 30 may also be comprised of one of the estrogen types and an oil selected from the group consisting of: soybean oil, corn oil, olive oil, castor oil, sesame oil, light mineral oil, heavy mineral oil, coconut oil, and canola oil, or other biocompatible oils.

1. An implantable sustained release estrogen delivery device comprising:
a reservoir housing;
a nanoporous membrane;
a formulation of estrogen.

2. (canceled)

3. (canceled)

4. (canceled)

5. (canceled)

6. The implantable sustained release estrogen delivery device of claim 1 wherein the nanoporous membrane is made of porous titanium with average pore size between 3 nm and 500 nm.

7. The implantable sustained release estrogen delivery device of claim 1 wherein the nanoporous membrane is made of porous polyetheretherketone (PEEK) with average pore size between 3 nm and 500 nm.

8. The implantable sustained release estrogen delivery device of claim 1 wherein the nanoporous membrane is made of porous 316L stainless steel with average pore size between 3 nm and 500 nm.

9. The implantable sustained release estrogen delivery device of claim 1 wherein the nanoporous membrane is made of porous aluminum oxide with average pore size between 3 nm and 500 nm.

10. The implantable sustained release estrogen delivery device of claim 1 wherein the nanoporous membrane is made of porous zirconium oxide with average pore size between 3 nm and 500 nm.

11. The implantable sustained release estrogen delivery device of claim 1 wherein the nanoporous membrane is made of porous polyethersulfone with average pore size between 3 nm and 500 nm.

12. The implantable sustained release estrogen delivery device of claim 1 wherein the nanoporous membrane is made of porous nylon with average pore size between 3 nm and 500 nm.

13. The implantable sustained release drug delivery device of claim 1 wherein the nanoporous membrane is made of porous polyester, polyethylene terephthalate, with average pore size between 3 nm and 500 nm.

14. The implantable sustained release drug delivery device of claim 1 wherein the nanoporous membrane is made of porous polycarbonate, with average pore size between 3 nm and 500 nm.

15. The implantable sustained release drug delivery device of claim 1 wherein the nanoporous membrane is made of porous cellulose acetate or cellulose nitrate or mixed cellulose, with average pore size between 3 nm and 500 nm.

16. The implantable sustained release estrogen delivery device of claim 1 wherein the formulation of estrogen is comprised of a formulation of estrogen and an oil.

17. The implantable sustained release estrogen delivery device of claim 1 wherein the formulation of estrogen is comprised of an estrogen compound selected from the following group: estrogen, estriol, estradiol, estradiol enanthate, ethinyl estradiol, estrone, or estrogen sulfate.

18. (canceled)

19. (canceled)

20. (canceled)

21. (canceled)

22. (canceled)

23. (canceled)

24. The implantable sustained release estrogen delivery device of claim 1 wherein the formulation of estrogen is comprised of an estrogen formulation and progesterone or progestin.

25. (canceled)

26. (canceled)

27. (canceled)

28. (canceled)

29. The implantable sustained release estrogen delivery device of claim 1 wherein an estrogen formulation including an oil the oil is selected from the group consisting of: soybean oil, corn oil, olive oil, castor oil, sesame oil, light mineral oil, heavy mineral oil, coconut oil, and canola oil, or other biocompatible oil.

30. The implantable sustained release estrogen delivery device of claim 1 wherein the formulation of estrogen is comprised an estrogen formulation and a testosterone formulation selected from the following group: testosterone, testosterone enanthate, testosterone decanoate, testosterone cypionate, testosterone isocaproate, testosterone phenylpropionate, testosterone propionate, or methyltestosterone.
